(19) United States
(12) Patent Application Publication
(10) Pub. No.: US 2022/0387399 A1
(43) Pub. Date: Dec. 8, 2022

(54) METHOD OF TREATING RNA REPEAT MEDIATED DISEASES WITH RNA REPEAT BINDING COMPOUND

(71) Applicant: AMO Pharma Ltd., Godalming (GB)

(72) Inventor: Michael SNAPE, Godalming (GB)

(73) Assignee: AMO Pharma Ltd., Godalming (GB)

(21) Appl. No.: 17/749,748

(22) Filed: May 20, 2022

Related U.S. Application Data
(60) Provisional application No. 63/191,531, filed on May 21, 2021.

(51) Int. Cl. A61K 31/433; A61P 43/00

(52) U.S. Cl. CPC A61K 31/433; A61P 43/00

(57) ABSTRACT

The use of 2,4-disubstituted thiadiazolidinone (TDZD) compounds, such as Tideglusib (4-Benzyl-2-naphthalen-1-yl-[1,2,4]thiadiazolidine-3,5-dione), in methods of inhibiting RNA molecules comprising abnormal trinucleotide repeats (such as CUG) in their sequences is provided. Such methods include methods of inhibiting RNA molecules having abnormal repeat sequences, as well as methods of treating and/or preventing diseases associated with the presence of RNA molecules having abnormal repeat sequences, such as myotonic dystrophy type 1 (DM1).

### SEQUENCE LISTING

A sequence listing in electronic (ASCII text file) format is filed with this application and incorporated herein by reference. The name of the ASCII text file is “2022_0872A_ST25.txt”; the file was created on May 17, 2022; the size of the file is 1296 bytes.

### BACKGROUND OF THE INVENTION

The genome comprising sequences of deoxyribonucleic acid (DNA) is coded into corresponding sequences of ribonucleic acid (RNA). These RNA sequences serve the function of coding for proteins which in turn comprise sequences of amino acids. As well as this role in normal physiological function, RNA sequences are proposed to play a role in the pathogenesis of disease.

A number of disorders are associated with abnormal repeated sequences of DNA in the genome, which are coded to corresponding abnormal repeated sequences of RNA. Evidence has been provided to show these abnormal RNA repeats play a role in the pathogenesis of some diseases. These RNAs can lead to disease in two ways. If the genomic repeat sequences of DNA base pairs are in the coding region of the relevant gene, then the corresponding RNA will code for an expanded protein. Proteins containing expanded sequences of repeated amino acids may be prone to aggregation, and aggregated proteins can interfere with cellular function. If the repeat sequences of DNA base pairs are instead in non-coding regions, then the corresponding RNA repeat sequence may itself interfere with cellular function and so cause disease. Pathogenic RNA repeat sequences that do not code for proteins commonly form hairpin structures that sequester proteins, for example.

Diseases that result from RNA repeat sequences allied to coding regions that are translated into expanded proteins may include Huntington's Disease and amyotrophic lateral sclerosis (ALS). In Huntington's Disease, an expanded CAG repeat occurs in the coding region of the Huntingtin (HTT) gene. Similarly, the most common cause of ALS is a hexanucleotides GGGGCC repeat. Expanded CAG RNA repeats are also implicated in spinocerebellar ataxia (SCA) types 1 through 20. A further neurodegenerative disorder is FXTAS in which a CGG trinucleotide repeat leads to RNA mediated toxicity. Myotonic dystrophy type 1 (DM1) is an incurable neuromuscular disorder caused by an expanded CTG repeat in the 3′ untranslated region (UTR) of the dystrophia myotonica protein kinase (DMPK) gene that is transcribed into RNA, yielding RNA molecules containing r(CUG)^{exp}. This CUG RNA repeat sequesters proteins such as Muscleblind (MBNL1), which causes pre-mRNA splicing defects, as well as CUGBP-1 and GSK3β. Similarly, myotonic dystrophy type 2 (DM2) is caused by an expanded CCUG RNA repeat.

Myotonic dystrophy type 1 (DM1) may be divided into subsets. The CUG repeat RNA that causes the disorder is associated with symptoms once the RNA contains more than 50 repeats. Most affected individuals with an RNA containing around 150 to 600 repeats have an onset of symptoms in adulthood i.e. have Adult Onset DM1. Individuals with larger RNA repeats containing for example 1000 repeats have an earlier onset subtype of DM1 that can be identified as causing symptoms at birth i.e. Congenital Onset DM1. Congenital Onset DM1 has very severe symptoms, typically being life threatening so that less than half of individuals with this form live to adulthood (Reardon et al., 1996, Arch Dis Child 68:177).

Interestingly, myotonic dystrophy type 1 (DM1) is caused by a CUG repeat RNA sequence that can be modified. The literature teaches that when the CUG repeat RNA causative for DM1 contains interruptions by non-CUG repeats the clinical phenotype of DM1 patients is modified (Peric et al., 2022, Int J Mol Sci. 23:354; Braida et al., 2010, Hum Mol Genet 19:1399). For example, the presence of CAG, CCG or CTC repeats, interrupting the sequence of the DM1 CUG repeat, leads to reductions in severity of the clinical symptoms of DM1 (see for example Wenninger et al., 2021, Neurol Genet 7, e572). Conversely, patients with congenital onset DM1, who have the most severe symptoms have never been shown to display interrupting non-CUG sequences in their CUG repeat RNA.

Interference with translation of repeats in coding regions of genes may result in therapeutic benefit via prevention of generation of the corresponding aberrant protein. Direct toxicity of expanded RNA repeats may also be mitigated by therapeutics binding directly to the RNA repeat and interfering with the ability of the repeat to bind proteins. These therapeutic approaches have been attempted by use of antisense oligonucleotides and low molecular weight pharmaceuticals. Antisense oligonucleotides have the advantage of great selectivity but may have disadvantages in terms of tissue penetration when administered to human subjects. For example, antisense oligonucleotides do not commonly penetrate brain when administered systemically. Conversely, low molecular weight pharmaceuticals may readily enter brain and muscle tissue when administered orally. The development of new therapeutic approaches is needed and the present invention is directed to these and other important goals.

### SUMMARY OF THE INVENTION

The present invention is generally directed to the use of 2,4-disubstituted thiadiazolidinone (TDZD) compounds and related analogs thereof in methods of inhibiting RNA molecules comprising abnormal trinucleotide repeats in their sequences. As discussed in detail below, the inventors found that TDZD compounds, such as Tideglusib (4-Benzyl-2-naphthalen-1-yl-[1,2,4]thiadiazolidine-3,5-dione), bind with specificity to RNA molecules comprising abnormal CUG repeats in their sequences. Thus, the present invention is directed to methods that are based on this discovery. The methods of the invention include, inter alia, methods of inhibiting RNA molecules having abnormal repeat sequences, as well as methods of treating and/or preventing diseases associated with the presence of RNA molecules having abnormal repeat sequences.

In a first embodiment, the present invention is directed to a method of treating a disease associated with a RNA molecule having an abnormal repeat sequence in a subject, where the method comprises administering to a subject in need thereof a therapeutically-effective amount of a compound of Formula I or a pharmaceutically acceptable salt, prodrug or solvate thereof.

In a related embodiment, the invention is directed to a method of treating a subject having a disease associated with a RNA molecule having an abnormal repeat sequence, where the method comprises administering a therapeutically-effective amount of a compound of Formula I or a pharmaceutically acceptable salt, prodrug or solvate thereof to a subject having a disease associated with a RNA molecule having an abnormal repeat sequence.

In a further related embodiment, the invention is directed to use of a therapeutically-effective amount of a compound of Formula I or a pharmaceutically acceptable salt, prodrug or solvate thereof in the treatment of a subject having a disease associated with a RNA molecule having an abnormal repeat sequence.

A further related embodiment, the invention is directed to use of a compound of Formula I or a pharmaceutically acceptable salt, prodrug or solvate thereof in the manufacture of a medicament for the treatment of a subject having a disease associated with a RNA molecule having an abnormal repeat sequence.

In a second embodiment, the invention is directed to a method of treating a subject suspected of having a disease associated with a RNA molecule having an abnormal repeat sequence, where the method comprises administering to a subject suspected of having a disease associated with a RNA molecule having an abnormal repeat sequence a therapeutically-effective amount of a compound of Formula I or a pharmaceutically acceptable salt, prodrug or solvate thereof.

A related embodiment, the invention is directed to use of a therapeutically-effective amount of a compound of Formula I or a pharmaceutically acceptable salt, prodrug or solvate thereof in the treatment of a subject suspected of having a disease associated with a RNA molecule having an abnormal repeat sequence.

A further related embodiment, the invention is directed to use of a compound of Formula I or a pharmaceutically acceptable salt, prodrug or solvate thereof in the manufacture of a medicament for the treatment of a subject suspected of having a disease associated with a RNA molecule having an abnormal repeat sequence.

As used herein, a “subject suspected of having a disease associated with a RNA molecule having an abnormal repeat sequence” is a subject showing signs or symptoms of such a disease (such as DM1) but where the subject has not yet been tested for the presence of a RNA molecule having an abnormal repeat sequence or such test results have not yet been received.

In a third embodiment, the invention is directed to a method of preventing a disease associated with a RNA molecule having an abnormal repeat sequence in a subject, where the method comprises administering to a subject in need thereof a therapeutically-effective amount of a compound of Formula I or a pharmaceutically acceptable salt, prodrug or solvate thereof.

A related embodiment, the invention is directed to use of a therapeutically-effective amount of a compound of Formula I or a pharmaceutically acceptable salt, prodrug or solvate thereof in the prevention of a disease associated with a RNA molecule having an abnormal repeat sequence in a subject in need thereof.

A further related embodiment, the invention is directed to use of a therapeutically-effective amount of a compound of Formula I or a pharmaceutically acceptable salt, prodrug or solvate thereof in the manufacture of a medicament for the prevention of a disease associated with a RNA molecule having an abnormal repeat sequence.

A subject in need thereof includes a subject at risk of developing a disease associated with a RNA molecule having an abnormal repeat sequence. Such a subject may or may not have clinical symptoms of a disease associated with a RNA molecule having an abnormal repeat sequence. Such a subject may or may not have cells expressing a RNA molecule having an abnormal repeat sequence.

In a fourth embodiment, the invention is directed to a method of binding a RNA molecule having an abnormal repeat sequence with a compound of Formula I or a pharmaceutically acceptable salt, prodrug or solvate thereof, where the method comprises contacting a RNA molecule having an abnormal repeat sequence with a compound of Formula I or a pharmaceutically acceptable salt, prodrug or solvate thereof.

The binding may be specific or non-specific binding, temporary or permanent. The RNA molecule may bind the compound, or the compound may bind the RNA molecule. The contacting may be in vitro, ex vivo or in vivo.

In a fifth embodiment, the invention is directed to a method of inhibiting a RNA molecule having an abnormal repeat sequence with a compound of Formula I or a pharmaceutically acceptable salt, prodrug or solvate thereof, where the method comprises contacting a RNA molecule having an abnormal repeat sequence with a compound of Formula I or a pharmaceutically acceptable salt, prodrug or solvate thereof.

The inhibiting may be partial or complete, temporary or permanent. The inhibiting may inhibit an activity of the RNA molecule. The inhibiting may inhibit binding of the RNA molecule to another molecule. The inhibiting may inhibit binding of the RNA molecule by another molecule.

In a sixth embodiment, the invention is directed to a method for inducing degradation of a RNA molecule having an abnormal repeat sequence, where the method comprises contacting a RNA molecule having an abnormal repeat sequence with a compound of Formula I or a pharmaceutically acceptable salt, prodrug or solvate thereof.

The degradation may be partial or complete.

In a seventh embodiment, the invention is directed to a method of inhibiting a RNA molecule having an abnormal repeat sequence in a biological sample with a compound of Formula I, where the method comprises contacting the biological sample with a compound of Formula I or a pharmaceutically acceptable salt, prodrug or solvate thereof.

The biological sample may be cell cultures or extracts thereof, preparations of an enzyme suitable for in vitro assay, biopsied material obtained from a mammal or extracts thereof, and blood, saliva, urine, faeces, semen, tears, or other body fluids or extracts thereof. Thus, in one aspect, the invention is directed to the use of compounds of Formula I as reactives for biological assays, in particular as a reactive for RNA molecules having abnormal CUG repeat sequence.

In each of the embodiments of the invention, the compound of Formula I is a compound encompassed by the following formula and as further defined herein:

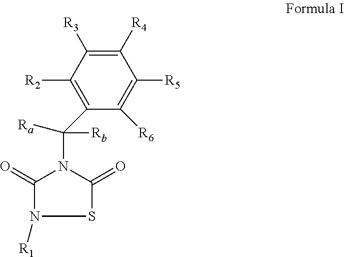

wherein:

R₁ is an organic group having at least 8 atoms selected from C or O, which is not linked directly to the N through a —C(O)— and comprising at least an aromatic ring;

Rₐ, R_{b}, R₂, R₃, R₄, R₅, R₆ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₇, —C(O)OR₇, —C(O)NR₇R₈—C═NR₇, —CN, —OR₇, —OC(O)R₇, —S(O)ₜ—R₇, —NR₇R₈, —NR₇C(O)R₈, —NO₂, —N═CR₇R₈ or halogen;

t is 0, 1, 2 or 3;

R₇ and R₈ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;

wherein Rₐ and R_{b} together can form a group ═O, and wherein any pair Rₐ R₂, R₂ R₃, R₃ R₄, R₄ R₅, R₅ R₆, R₆ R_{b}, or R₇R₈ can form together a cyclic substituent;

or a pharmaceutically acceptable salt, prodrug or solvate thereof.

In certain embodiments, the compound of Formula I is one of the following compounds:

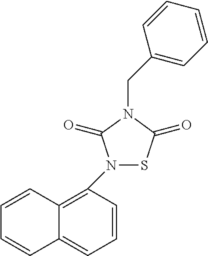 [SMILES: O=c1sn(-c2cccc3ccccc23)c(=O)n1Cc1ccccc1]

4-Benzyl-2-naphthalen-1-yl-[1,2,4]thiadiazolidine-3,5-dione (Tideglusib)

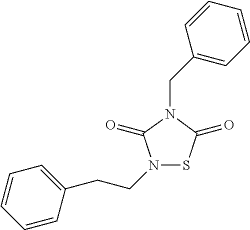 [SMILES: O=c1sn(CCc2ccccc2)c(=O)n1Cc1ccccc1]

4-Benzyl-2-phenethyl-[1,2,4]thiadiazolidine-3,5-dione

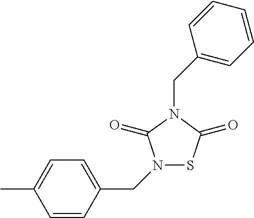 [SMILES: Cc1ccc(Cn2sc(=O)n(Cc3ccccc3)c2=O)cc1]

4-Benzyl-2-(4-methyl-benzyl)-[1,2,4]thiadiazolidine-3,5-dione

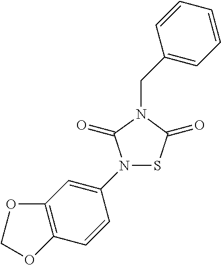 [SMILES: O=c1sn(-c2ccc3c(c2)OCO3)c(=O)n1Cc1ccccc1]

2-Benzo[1,3]dioxol-5-yl-4-benzyl-[1,2,4]thiadiazolidine-3,5-dione

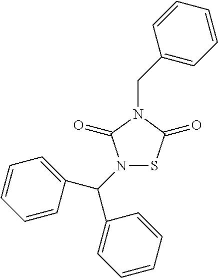 [SMILES: O=c1sn(C(c2ccccc2)c2ccccc2)c(=O)n1Cc1ccccc1]

4-Benzyl-2-diphenylmethyl-[1,2,4]-thiadiazolidine-3,5-dione

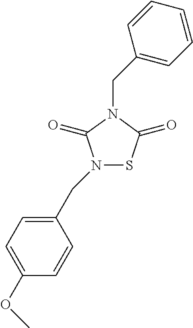 [SMILES: COc1ccc(Cn2sc(=O)n(Cc3ccccc3)c2=O)cc1]

4-Benzyl-2-(4-methoxy-benzyl)-[1,2,4]thiadiazolidine-3,5-dione

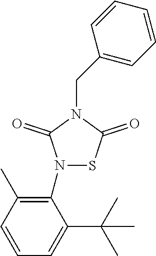 [SMILES: Cc1cccc(C(C)(C)C)c1-n1sc(=O)n(Cc2ccccc2)c1=O]

4-Benzyl-2-(2-tert-butyl-6-methyl-phenyl)-[1,2,4]thiadiazolidine-3,5-dione

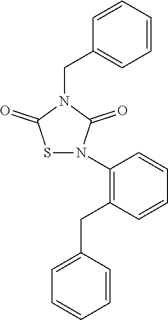 [SMILES: O=c1sn(-c2ccccc2Cc2ccccc2)c(=O)n1Cc1ccccc1]

4-Benzyl-2-(2-benzyl-phenyl)-[1,2,4]thiadiazolidine-3,5-dione

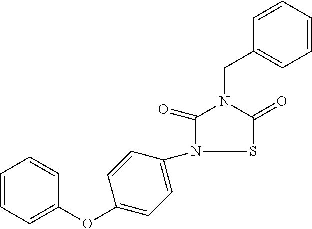 [SMILES: O=c1sn(-c2ccc(Oc3ccccc3)cc2)c(=O)n1Cc1ccccc1]

4-Benzyl-2-(4-phenoxy-phenyl)-[1,2,4]thiadiazolidine-3,5-dione

or a pharmaceutically acceptable salt, prodrug or solvate thereof.

In particular aspects of the invention, the compound of Formula I is Tideglusib (4-Benzyl-2-naphthalen-1-yl-[1,2,4]thiadiazolidine-3,5-dione) or a pharmaceutically acceptable salt, prodrug or solvate thereof.

As used herein, a “disease associated with a RNA molecule having an abnormal repeat sequence” includes, but is not limited, to myotonic dystrophy type 1 (DM1), also known as Steinhert's Disease, whether of Congenital, Childhood or Adult Onset sub-type DM1, as well as Fuchs endothelial corneal dystrophy and spinocerebellar ataxia type 8.

As used herein, a “RNA molecule having an abnormal repeat sequence” includes RNA molecules comprising abnormal trinucleotide repeats in their sequences. A non-limiting example of such RNA molecules include RNA molecules comprising abnormal CUG nucleotide repeats in their sequences. The CUG nucleotide repeats are one or more consecutive and uninterrupted CUG nucleotide repeat sequences of at least 50 CUG nucleotide repeats. In certain embodiments, the RNA molecule comprising abnormal trinucleotide repeats forms an RNA hairpin structure.

The methods of the invention may be practiced by delivering a compound of Formula I or a pharmaceutically acceptable salt, prodrug or solvate thereof directly to a subject. However, in preferred aspects of the invention, such compounds will be in the form of a pharmaceutical composition comprising a compound of Formula I along with one or more pharmaceutically acceptable excipient, carrier, adjuvant and/or vehicle. In a particular aspect, the pharmaceutical composition is formulated for oral delivery.

In a particular aspect of the first embodiment set forth above, the invention is directed to a method of treating myotonic dystrophy type 1 (DM1) in a subject, where the method comprises administering to a subject in need thereof a therapeutically-effective amount of Tideglusib (4-Benzyl-2-naphthalen-1-yl-[1,2,4]thiadiazolidine-3,5-dione) or a pharmaceutically acceptable salt, prodrug or solvate thereof. The DM1 may be Adult Onset DM1 or Congenital Onset DM1.

In a related particular aspect, the invention is directed to a method of treating a subject having DM1, where the method comprises administering a therapeutically-effective amount of Tideglusib (4-Benzyl-2-naphthalen-1-yl-[1,2,4]thiadiazolidine-3,5-dione) or a pharmaceutically acceptable salt, prodrug or solvate thereof to a subject having DM1. The DM1 may be Adult Onset DM1 or Congenital Onset DM1.

### BRIEF DESCRIPTION OF THE DRAWINGS

FIG. 1 shows binding of Tideglusib to an RNA construct containing CUG repeats of the sequence Cy5gggagaggguuuaaucugcugcugcugcugcuguacgaaaguacugcugcugcugcugcugauuggauccgcaagg3 (SEQ ID NO: 1) as assayed using a MicroScale Thermophoresis (MST) Fluorescence binding assay. The K_{D} of binding by Tideglusib was determined to be 19.7 nM.

FIG. 2 shows binding of Tideglusib to an RNA construct containing CAG repeats of the sequence Cy5gggagaggguuuaaucagcagcagcagcagcaguacgaaaguacagcagcagcagcagcagauuggauccgcaagg3 (SEQ ID NO:2) as assayed using a MicroScale Thermophoresis (MST) Fluorescence binding assay. No reliable of binding by Tideglusib could be determined.

FIG. 3 shows binding of the main metabolite of Tideglusib (4-Benzyl-2-naphthalen-1-yl-[1,2,4]thiadiazolidine-3,5-dione) to an RNA construct containing CUG repeats of the sequence Cy5gggagaggguuuaaucugcugcugcugcugcuguacgaaaguacugcugcugcugcugcugauuggauccgcaagg3 (SEQ ID NO: 1) as assayed using a MicroScale Thermophoresis (MST) Fluorescence binding assay. No reliable binding of this compound could be determined.

FIG. 4 shows levels of GSK3β kinase protein as assayed by ELISA in lymphocytes extracted from Congenital Onset DM1 patients treated with placebo and Compound 1 (Tideglusib) at either 400 mg or 1000 mg. GSK3β levels were assayed at baseline (v2), after placebo treatment (v3), after six weeks of treatment (v4), and twelve weeks of treatment (v9). Compound administration had no effect.

FIG. 5 shows levels of phosphorylated Akt (pAkt) kinase protein as assayed by ELISA in lymphocytes extracted from Congenital Onset DM1 patients treated with placebo and Compound 1 (Tideglusib) at either 400 mg or 1000 mg. GSK3β levels were assayed at baseline (v2), after placebo treatment (v3), after six weeks of treatment (v4), and twelve weeks of treatment (v9). Compound administration had no effect.

FIG. 6 shows the clinical benefit of 400 mg or 1000 mg Tideglusib in patients having DM1 as assessed using the Clinical Global Impression of Improvement measurement scale.

FIG. 7 shows percent reduction in levels of phosphorylated Akt (pAkt), ERK (pERK) and JNK (pJNK) seen in lymphocytes from pediatric subjects with Autism Spectrum Disorder at the end of treatment for 12 weeks with placebo (N=42) or compound 1 (Tideglusib) titrated from 400 mg to 1000 mg over six weeks followed by six week 1000 mg oral once per day (N=41). Tideglusib inhibited phosphorylation of Akt (p<0.0001), which is a phosphorylation target of GSK3β, but did not inhibit phosphorylation of ERK or JNK, which are not phosphorylated by GSK3β.

FIG. 8 shows ELISA assay of GSK3β levels in lymphocytes of N=16 subjects with Congenital or Childhood Onset Myotonic Dystrophy Type 1 compared to levels seen in subjects who do not have Myotonic Dystrophy. GSK3β levels are five times increased in Myotonic Dystrophy.

FIG. 9 shows binding of Tideglusib to an RNA construct containing CUG repeats with interspersed interrupting non CUG repeats (GGC and CUC) of the sequence 5′-Cy5-gggagaggguuuaaucugcugcugcugcugccgcugcugcugcugcugcuccugcugcugcugcugccgcugcugcugcugcugcucu acgaaaguagcugcugcugcugcuggcgcugcugcugcugcugcggcugcugcugcugcuggcgcugcugcugcugcugcgauugga uccgcaagg-3 (SEQ ID NO:3) as assayed using a MicroScale Thermophoresis (MST) Fluorescence binding assay. The K_{D} of binding by Tideglusib was determined to be 3,200 nM.

### DETAILED DESCRIPTION OF THE INVENTION

Tideglusib (4-Benzyl-2-naphthalen-1-yl-[1,2,4]thiadiazolidine-3,5-dione) is a molecule that is known to penetrate muscle, brain and other organ systems when given orally. It has been shown to confer therapeutic benefit in myotonic dystrophy type 1 (DM1) and to cause fragmentation of the repeat CUG RNA pathogenic for this disorder. Previous explanations of the effect of Tideglusib on both efficacy in patients and fragmentation of CUG RNA repeats are implied to be a result of inhibition of GSK3β by substituted thiadiazolidines (Jones et al., PNAS 112(26):8041-8045 (2015)). However, the dose administration schedule used in clinical testing is not compatible with inhibition of this kinase in DM1 myotonic dystrophy.

It was only through the efforts of the present inventors that it was discovered Tideglusib binds directly to RNA molecules having abnormal repeat sequences, such as the CUG repeat RNA molecules associated with DM1. The present invention is based on this important discovery.

Methods of Treatment and/or Prevention

As summarized above, and discussed in further detail below, the invention includes methods of treating and/or preventing diseases associated with RNA molecules having abnormal repeat sequences, such as the CUG repeat RNA molecules associated with DM1.

Thus, the invention is directed to a method of treating a disease associated with a RNA molecule having an abnormal repeat sequence in a subject, where the method comprises administering to a subject in need thereof a therapeutically-effective amount of a compound of Formula I or a pharmaceutically acceptable salt, prodrug or solvate thereof.

In a related embodiment, the invention is directed to a method of treating a subject having a disease associated with a RNA molecule having an abnormal repeat sequence, where the method comprises administering a therapeutically-effective amount of a compound of Formula I or a pharmaceutically acceptable salt, prodrug or solvate thereof to a subject having a disease associated with a RNA molecule having an abnormal repeat sequence.

In a further related embodiment, the invention is directed to use of a therapeutically-effective amount of a compound of Formula I or a pharmaceutically acceptable salt, prodrug or solvate thereof in the treatment of a subject having a disease associated with a RNA molecule having an abnormal repeat sequence.

A further related embodiment, the invention is directed to use of a compound of Formula I or a pharmaceutically acceptable salt, prodrug or solvate thereof in the manufacture of a medicament for the treatment of a subject having a disease associated with a RNA molecule having an abnormal repeat sequence.

The invention is also directed to a method of treating a subject suspected of having a disease associated with a RNA molecule having an abnormal repeat sequence, where the method comprises administering to a subject suspected of having a disease associated with a RNA molecule having an abnormal repeat sequence a therapeutically-effective amount of a compound of Formula I or a pharmaceutically acceptable salt, prodrug or solvate thereof.

A related embodiment, the invention is directed to use of a therapeutically-effective amount of a compound of Formula I or a pharmaceutically acceptable salt, prodrug or solvate thereof in the treatment of a subject suspected of having a disease associated with a RNA molecule having an abnormal repeat sequence.

A further related embodiment, the invention is directed to use of a compound of Formula I or a pharmaceutically acceptable salt, prodrug or solvate thereof in the manufacture of a medicament for the treatment of a subject suspected of having a disease associated with a RNA molecule having an abnormal repeat sequence.

As used herein, a “subject suspected of having a disease associated with a RNA molecule having an abnormal repeat sequence” is a subject showing signs or symptoms of such a disease (such as DM1) but where the subject has not yet been tested for the presence of a RNA molecule having an abnormal repeat sequence or such test results have not yet been received.

The invention is further directed to a method of preventing a disease associated with a RNA molecule having an abnormal repeat sequence in a subject, where the method comprises administering to a subject in need thereof a therapeutically-effective amount of a compound of Formula I or a pharmaceutically acceptable salt, prodrug or solvate thereof.

A related embodiment, the invention is directed to use of a therapeutically-effective amount of a compound of Formula I or a pharmaceutically acceptable salt, prodrug or solvate thereof in the prevention of a disease associated with a RNA molecule having an abnormal repeat sequence in a subject in need thereof.

A further related embodiment, the invention is directed to use of a therapeutically-effective amount of a compound of Formula I or a pharmaceutically acceptable salt, prodrug or solvate thereof in the manufacture of a medicament for the prevention of a disease associated with a RNA molecule having an abnormal repeat sequence.

A subject in need thereof includes a subject at risk of developing a disease associated with a RNA molecule having an abnormal repeat sequence. Such a subject may or may not have clinical symptoms of a disease associated with a RNA molecule having an abnormal repeat sequence. Such a subject may or may not have cells expressing a RNA molecule having an abnormal repeat sequence.

As used herein, the “subject” is a human, a non-human primate, bird, horse, cow, goat, sheep, a companion animal, such as a dog, cat or rodent, or other mammal.

Methods of Binding, Inhibiting and/or Degrading RNA Molecules

As summarized above, and discussed in further detail below, the invention also includes methods related to binding, inhibiting and/or degrading RNA molecules having abnormal repeat sequences, such as the CUG repeat RNA molecules associated with DM1. Such methods may be practiced in vitro, ex vivo or in vivo.

Thus, the invention is directed to a method of binding a RNA molecule having an abnormal repeat sequence with a compound of Formula I or a pharmaceutically acceptable salt, prodrug or solvate thereof, where the method comprises contacting a RNA molecule having an abnormal repeat sequence with a compound of Formula I or a pharmaceutically acceptable salt, prodrug or solvate thereof. The binding may be specific or non-specific binding, temporary or permanent. The RNA molecule may bind the compound, or the compound may bind the RNA molecule. The contacting may be in vitro, ex vivo or in vivo.

The method of binding a RNA molecule having an abnormal repeat sequence with a compound of Formula I or a pharmaceutically acceptable salt, prodrug or solvate thereof can be used as a diagnostic or companion diagnostic in diagnosing a disease associated with a RNA molecule having an abnormal repeat sequence, such as DM1 and the other diseases mentioned herein. For example, a compound of Formula I, such as Tideglusib, can be used to screen for RNA molecules having an abnormal repeat sequence, such as a RNA molecule comprising abnormal CUG nucleotide repeats, in a biological sample. As defined herein, the CUG nucleotide repeats are consecutive and uninterrupted CUG nucleotide repeat sequences of at least 10 CUG repeats. The CUG nucleotide repeats may also be consecutive and uninterrupted CUG nucleotide repeats of at least 25, 30, 35, 40, 45, 50, 55, 60, 65, 70, 75, 80, 85, 90, 95, 100 or more CUG nucleotide repeats. Thus, the present invention is also directed to a method for diagnosing DM1 or confirming a diagnosis of DM1 in a subject where the method comprises screening a biological sample from a subject for a RNA molecule having an abnormal repeat sequence by contacting the biological sample with a compound of Formula I or a pharmaceutically acceptable salt, prodrug or solvate thereof and detecting binding of such a RNA molecule by the compound of Formula I.

The invention is also directed to a method of inhibiting a RNA molecule having an abnormal repeat sequence with a compound of Formula I or a pharmaceutically acceptable salt, prodrug or solvate thereof, where the method comprises contacting a RNA molecule having an abnormal repeat sequence with a compound of Formula I or a pharmaceutically acceptable salt, prodrug or solvate thereof. The inhibiting may be partial or complete, temporary or permanent. The inhibiting may inhibit an activity of the RNA molecule. The inhibiting may inhibit binding of the RNA molecule to another molecule. The inhibiting may inhibit binding of the RNA molecule by another molecule.

The invention is further directed to a method for inducing degradation of a RNA molecule having an abnormal repeat sequence, where the method comprises contacting a RNA molecule having an abnormal repeat sequence with a compound of Formula I or a pharmaceutically acceptable salt, prodrug or solvate thereof. The degradation may be partial or complete.

In another aspect, the invention relates to inhibiting RNA molecules having abnormal CUG repeat sequence in a biological sample with a compound of Formula I. This method comprises contacting the biological sample with a compound of Formula I or a pharmaceutically acceptable salt, prodrug or solvate thereof. The term “biological sample”, as used herein, includes, but is not limited to, cell cultures or extracts thereof, preparations of an enzyme suitable for in vitro assay, biopsied material obtained from a mammal or extracts thereof, and blood, saliva, urine, faeces, semen, tears, or other body fluids or extracts thereof. Thus, in one aspect, the invention is directed to the use of compounds of Formula I as reactives for biological assays, in particular as a reactive for RNA molecules having abnormal CUG repeat sequence.

### 2,4-Disubstituted Thiadiazolidinone (TDZD) Compounds

As indicated above, the present invention is directed to use of 2,4-disubstituted thiadiazolidinone (TDZD) compounds in the methods of the invention. Exemplary TDZD compounds that may be used in methods of the invention are the compounds defined by Formula I:

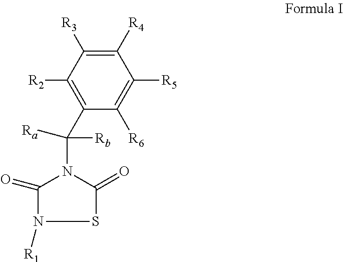

wherein:

R₁ is an organic group having at least 8 atoms selected from C or O, which is not linked directly to the N through a —C(O)— and comprising at least an aromatic ring;

Rₐ, R_{b}, R₂, R₃, R₄, R₅, R₆ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₇, —C(O)OR₇, —C(O)NR₇R₈—C═NR₇, —CN, —OR₇, —OC(O)R₇, —S(O)ₜ—R₇, —NR₇R₈, —NR₇C(O)R₈, —NO₂, —N═CR₇R₈ or halogen;

t is 0, 1, 2 or 3;

R₇ and R₈ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;

wherein Rₐ and R_{b} together can form a group ═O, and wherein any pair Rₐ R₂, R₂ R₃, R₃ R₄, R₄ R₅, R₅ R₆, R₆ R_{b}, or R₇R₈ can form together a cyclic substituent;

or a pharmaceutically acceptable salt, prodrug or solvate thereof.

R₁ comprises an aromatic group, this improves the stability properties. In an embodiment, R₁ has at least 10 aromatic carbons. Alternatively, compounds with excellent activity are obtained with electron donating groups on the aromatic ring such as alkoxyl or methylendioxy.

Although R₁ can be linked to the TDZD through any group as long as it is not —C(O)— (because of degradation and poor stability in plasma), it is preferred that the aromatic group is directly linked to the N of the thiadiazolidine.

In a particular embodiment, compounds in which R₁ is a naphthyl group are exemplary, most exemplary is where R₁ is an α-naphthyl group. When R₁ is α-naphthyl, it is preferred that it is an unsubstituted α-naphthyl.

Representative substituents that can be used as R₁ are the following:

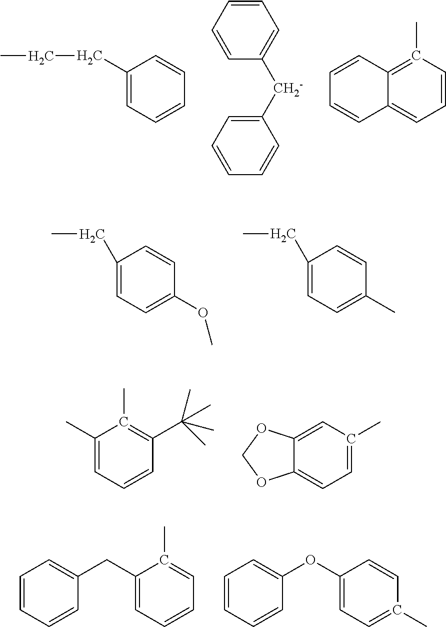

In another embodiment, R₂, R₃, R₄ R₅, R₆ are independently selected from hydrogen, substituted or unsubstituted alkyl, COR₇, —C(O)OR₇, —OR₇, —NR₇R₈, or halogen. In an exemplary aspect, the substituent at position 4 is unsubstituted benzyl group. Concerning the substituent at position 4 of the TDZD, Rₐ and R_{b} may be both H.

Representative compounds of Formula I are the following:

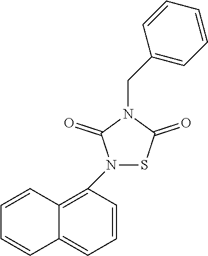 [SMILES: O=c1sn(-c2cccc3ccccc23)c(=O)n1Cc1ccccc1]

4-Benzyl-2-naphthalen-1-yl-[1,2,4]thiadiazolidine-3,5-dione (Tideglusib)

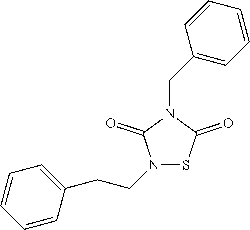 [SMILES: O=c1sn(CCc2ccccc2)c(=O)n1Cc1ccccc1]

4-Benzyl-2-phenethyl-[1,2,4]thiadiazolidine-3,5-dione

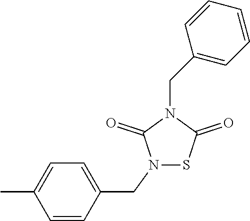 [SMILES: Cc1ccc(Cn2sc(=O)n(Cc3ccccc3)c2=O)cc1]

4-Benzyl-2-(4-methyl-benzyl)-[1,2,4]thiadiazolidine-3,5-dione

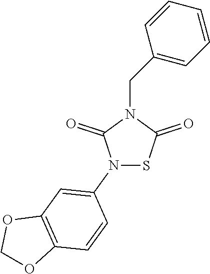 [SMILES: O=c1sn(-c2ccc3c(c2)OCO3)c(=O)n1Cc1ccccc1]

2-Benzo[1,3]dioxol-5-yl-4-benzyl-[1,2,4]thiadiazolidine-3,5-dione

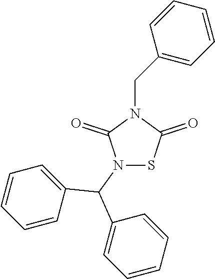 [SMILES: O=c1sn(C(c2ccccc2)c2ccccc2)c(=O)n1Cc1ccccc1]

4-Benzyl-2-diphenylmethyl-1,2,4-thiadiazolidine-3,5-dione

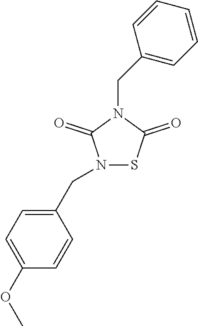 [SMILES: COc1ccc(Cn2sc(=O)n(Cc3ccccc3)c2=O)cc1]

4-Benzyl-2-(4-methoxy-benzyl)-[1,2,4]thiadiazolidine-3,5-dione

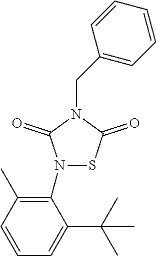 [SMILES: Cc1cccc(C(C)(C)C)c1-n1sc(=O)n(Cc2ccccc2)c1=O]

4-Benzyl-2-(2-tert-butyl-6-methyl-phenyl)-[1,2,4]thiadiazolidine-3,5-dione

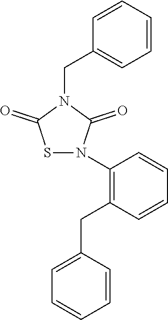 [SMILES: O=c1sn(-c2ccccc2Cc2ccccc2)c(=O)n1Cc1ccccc1]

4-Benzyl-2-(2-benzyl-phenyl)-[1,2,4]thiadiazolidine-3,5-dione

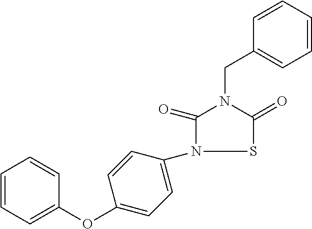 [SMILES: O=c1sn(-c2ccc(Oc3ccccc3)cc2)c(=O)n1Cc1ccccc1]

4-Benzyl-2-(4-phenoxy-phenyl)-[1,2,4]thiadiazolidine-3,5-dione, or a pharmaceutically acceptable salt, prodrug or solvate thereof.

In particular aspects of the invention, the compound of Formula I is Tideglusib (4-Benzyl-2-naphthalen-1-yl-[1,2,4]thiadiazolidine-3,5-dione) or a pharmaceutically acceptable salt, prodrug or solvate thereof.

In the definition of compounds of Formula I, the terms have the following indicated meanings.

“Alkyl” refers to a straight or branched hydrocarbon chain radical consisting of carbon and hydrogen atoms, containing no saturation, having one to eight carbon atoms, and which is attached to the rest of the molecule by a single bond, e.g., methyl, ethyl, n-propyl, i-propyl, n-butyl, t-butyl, n-pentyl, etc. Alkyl radicals may be optionally substituted by one or more substituents such as halo, hydroxy, alkoxy, carboxy, cyano, carbonyl, acyl, alkoxycarbonyl, amino, nitro, mercapto and alkylthio.

“Alkoxy” refers to a radical of the formula —ORₐ where Rₐ is an alkyl radical as defined above, e.g., methoxy, ethoxy, propoxy, etc.

“Alkoxycarbonyl” refers to a radical of the formula —C(O)ORₐ where Rₐ is an alkyl radical as defined above, e.g., methoxycarbonyl, ethoxycarbonyl, propoxycarbonyl, etc.

“Alkylthio” refers to a radical of the formula —SRₐ where Rₐ is an alkyl radical as defined above, e.g., methylthio, ethylthio, propylthio, etc.

“Amino” refers to a radical of the formula —NH₂, —NHRₐ or —NRₐR_{b}, wherein Rₐ and R_{b} are as defined above.

“Aryl” refers to a phenyl, naphthyl, indenyl, fenanthryl or anthracyl radical, preferably phenyl or naphthyl radical. The aryl radical may be optionally substituted by one or more substituents such as hydroxy, mercapto, halo, alkyl, phenyl, alkoxy, haloalkyl, nitro, cyano, dialkylamino, aminoalkyl, acyl and alkoxycarbonyl, as defined herein.

“Aralkyl” refers to an aryl group linked to an alkyl group. Preferred examples include benzyl and phenethyl.

“Acyl” refers to a radical of the formula —C(O)—R_{c} and —C(O)—Rₐ where Rₑ is an alkyl radical as defined above and R_{d} is an aryl radical as defined above, e.g., acetyl, propionyl, benzoyl, and the like.

“Aroylalkyl” refers to an alkyl group substituted with —Rₐ—C(O)—Rₐ, wherein Rₐ is an alkyl radical. Preferred examples include benzoylmethyl.

“Carboxy” refers to a radical of the formula —C(O)OH.

“Cycloalkyl” refers to a stable 3- to 10-membered monocyclic or bicyclic radical which is saturated or partially saturated, and which consist solely of carbon and hydrogen atoms. Unless otherwise stated specifically in the specification, the term “cycloalkyl” is meant to include cycloalkyl radicals which are optionally substituted by one or more such as alkyl, halo, hydroxy, amino, cyano, nitro, alkoxy, carboxy and alkoxycarbonyl.

“Fused aryl” refers to an aryl group, especially a phenyl or heteroaryl group, fused to another ring.

“Halo” refers to bromo, chloro, iodo or fluoro.

“Haloalkyl” refers to an alkyl radical, as defined above, that is substituted by one or more halo radicals, as defined above, e.g., trifluoromethyl, trichloromethyl, 2,2,2-trifluoroethyl, 1-fluoromethyl-2-fluoroethyl, and the like.

“Heterocycle” refers to a heterocyclyl radical. The heterocycle refers to a stable 3- to 15-membered ring which consists of carbon atoms and from one to five heteroatoms selected from the group consisting of nitrogen, oxygen, and sulfur, preferably a 4- to 8-membered ring with one or more heteroatoms, more preferably a 5- or 6-membered ring with one or more heteroatoms. For the purposes of this invention, the heterocycle may be a monocyclic, bicyclic or tricyclic ring system, which may include fused ring systems; and the nitrogen, carbon or sulfur atoms in the heterocyclyl radical may be optionally oxidised; the nitrogen atom may be optionally quaternized; and the heterocyclyl radical may be partially or fully saturated or aromatic. Examples of such heterocycles include, but are not limited to, azepines, benzimidazole, benzothiazole, furan, isothiazole, imidazole, indole, piperidine, piperazine, purine, quinoline, thiadiazole, tetrahydrofuran.

References herein to substituted groups in the compounds of Formula I refer to the specified moiety that may be substituted at one or more available positions by one or more suitable groups, e.g., halogen such as fluoro, chloro, bromo and iodo; cyano; hydroxyl; nitro; azido; alkanoyl such as a C1-6 alkanoyl group such as acyl and the like; carboxamido; alkyl groups including those groups having 1 to about 12 carbon atoms or from 1 to about 6 carbon atoms and more preferably 1-3 carbon atoms; alkenyl and alkynyl groups including groups having one or more unsaturated linkages and from 2 to about 12 carbon or from 2 to about 6 carbon atoms; alkoxy groups having one or more oxygen linkages and from 1 to about 12 carbon atoms or 1 to about 6 carbon atoms; aryloxy such as phenoxy; alkylthio groups including those moieties having one or more thioether linkages and from 1 to about 12 carbon atoms or from 1 to about 6 carbon atoms; alkylsulfinyl groups including those moieties having one or more sulfinyl linkages and from 1 to about 12 carbon atoms or from 1 to about 6 carbon atoms; alkylsulfonyl groups including those moieties having one or more sulfonyl linkages and from 1 to about 12 carbon atoms or from 1 to about 6 carbon atoms; aminoalkyl groups such as groups having one or more N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

Unless otherwise stated, the compounds of Formula I are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon or ¹⁵N-enriched nitrogen are within the scope of this invention.

The term “pharmaceutically acceptable salt, prodrug or solvate thereof” refers to any pharmaceutically acceptable salt, ester, solvate, or any other compound which, upon administration to the recipient, is capable of providing (directly or indirectly) a compound as described herein. The preparation of salts, prodrugs and derivatives can be carried out by methods known in the art. For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glucamine and basic aminoacids salts.

Particularly favoured derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is a prodrug of a compound of Formula I is within the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well-known methods of producing a prodrug of a given acting compound are known to those skilled in the art and can be found, e.g., in Krogsgaard-Larsen et al. “Textbook of Drug Design and Discovery” Taylor & Francis (April 2002).

The compounds of Formula I for use in the methods of the invention may be in crystalline form either as free compounds or as solvates (e.g. hydrates) and it is intended that both forms are within the scope of the present invention. Methods of solvation are generally known within the art. Suitable solvates are pharmaceutically acceptable solvates. In a particular embodiment the solvate is a hydrate. The compounds of Formula I or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of Formula I, or of its salts, solvates or prodrugs.

The compounds of Formula I may include enantiomers depending on the presence of chiral centres or isomers depending on the presence of multiple bonds (e.g. Z, E). The single isomers, enantiomers or diastereoisomers and mixtures thereof fall within the scope of the present invention.

The compounds of Formula I defined herein can be obtained by available synthetic procedures. Some examples of these procedures are described in WO 05/097117, WO 01/85685 and US 2003/0195238 and references cited therein. The contents of each of these documents are incorporated herein by reference in their entirety.

### RNA Repeat Mediated Diseases

As used herein, a “disease associated with a RNA molecule having an abnormal repeat sequence” includes, but is not limited, to myotonic dystrophy type 1 (DM1), also known as Steinhert's Disease, whether of Congenital, Childhood or Adults Onset sub-type DM1. Thus, the methods of the invention include methods for treating Congenital or Childhood Onset DM1, as well as method for treating Adult Onset DM1. Diseases associated with a RNA molecule having an abnormal repeat sequence also include Fuchs endothelial corneal dystrophy and spinocerebellar ataxia type 8. Each of these disorders and diseases is associated with CUG repeat RNA (Childs-Disney et al., 2012, Acs Chem Biol 7:856; Du et al., 2015, J Biol Chem 290:5979; Daughter et al., 2009, PLoS Genet 5:e1000600).

The compounds of Formula I, such as Tideglusib, can reverse clinical deficits in patients with CUG repeats, most particularly in those patients without non-CUG RNA repeat sequences interrupting their CUG RNA repeats, specifically Congenital Onset DM1 and Adult Onset DM1, where Adult Onset DM1 patients do not have interrupting non-CUG RNA repeat sequences.

### Abnormal RNA Molecules

As used herein, a “RNA molecule having an abnormal repeat sequence” includes RNA molecules comprising abnormal trinucleotide repeats in their sequences. As used herein, “abnormal” means a number of repeats that alters the normal functioning of the RNA molecule, i.e. the functioning of the RNA molecule that would be seen in a subject not having a disease that is a subject of the present invention. A non-limiting example of such RNA molecules include RNA molecules comprising abnormal CUG nucleotide repeats in their sequences. The CUG nucleotide repeats are consecutive and uninterrupted CUG nucleotide repeats of at least 10 CUG repeats. The CUG nucleotide repeats may also be consecutive and uninterrupted CUG nucleotide repeat sequences of at least 25, 30, 35, 40, 45, 50, 55, 60, 65, 70, 75, 80, 85, 90, 95, 100 or more CUG nucleotide repeats. In certain instances, the CUG nucleotide repeats may be interrupted by non-CUG nucleotides. In certain embodiments, the RNA molecule comprising abnormal trinucleotide repeats forms an RNA hairpin structure.

### Pharmaceutical Composition and Means of Delivery

The methods of the invention may be practiced by delivering a compound of Formula I or a pharmaceutically acceptable salt, prodrug or solvate thereof directly to a subject. However, in preferred aspects of the invention, such compounds will be in the form of a pharmaceutical composition comprising a compound of Formula I along with one or more pharmaceutically acceptable excipient, carrier, adjuvant and/or vehicle. Examples of pharmaceutical compositions include any solid (tablets, pills, capsules, granules etc.) or liquid (solutions, suspensions or emulsions) composition for oral, topical or parenteral administration. In a preferred embodiment the pharmaceutical compositions are in oral form.

Suitable dose forms for oral administration may be tablets and capsules and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in US Pharmacopoeias and similar reference texts.

Administration of the compounds or compositions to a subject may be by any suitable method, such as intravenous infusion, oral preparations, and intraperitoneal and intravenous administration. Oral administration is preferred because of the convenience for the patient and the chronic character of many of the diseases to be treated.

Generally, a therapeutically-effective amount of a compound of Formula I or a pharmaceutical composition comprising the compound is an amount sufficient to reduce one or more clinical symptoms of the disease, determined, for example, by a subject's doctor. The amount will also depend on the relative efficacy of the compound chosen, the severity of the disorder being treated and the weight of the sufferer. However, compounds will typically be administered once or more times a day for example 1, 2, 3 or 4 times daily, or once every 2, 3, 4 or 5 days, with typical total daily dose in the range of from 0.1 to 10,000 mg. The total daily dose may also range from 1 to 5000 mg, 10 to 2500 mg, 50 to 1500 mg, 100 to 1250 mg, or 300 to 1000 mg, administered once per day or once every two days. Suitable doses may also be defined as 100 mg, 200 mg, 300 mg, 400 mg, 500 mg, 600 mg, 700 mg, 800 mg, 900 mg, 1000 mg, 1100 mg, 1200 mg, 1300 mg, 1400 mg, or 1500 mg, administered once per day or once every two days The compounds and compositions of this invention may be used with other drugs to provide a combination therapy. The other drugs may form part of the same composition, or be provided as a separate composition for administration at the same time or at different time.

### EXAMPLES

The following examples are intended to further illustrate the invention. They should not be interpreted as a limitation of the scope of the invention as defined in the claims.

Tideglusib (4-benzyl-2-(naphthalen-1-yl)-1,2,4-thiadiazolidine-3,5-dione; Compound 1) is a well-tolerated thiadiazolidine compound that acts as a glycogen synthase kinase (isoform 3 beta) (GSK3β) inhibitor. It is known to penetrate muscle, brain and other organ systems when given orally. The compound was developed as an inhibitor of GSK3β, but it has also been shown to confer therapeutic benefit in DM1 myotonic dystrophy.

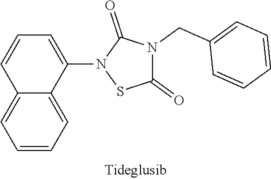 [SMILES: O=c1sn(-c2cccc3ccccc23)c(=O)n1Cc1ccccc1]

As demonstrated by the present inventors, Tideglusib acts by binding directly to RNA molecules comprising repeating CUG nucleotide triplets. This direct binding was determined using a MicroScale Thermophoresis (MST) Fluorescence binding assays with Tideglusib and an RNA hairpin containing CUG repeats. The RNA hairpin was modeled after the structure reported by Parkesh et al (J. Am. Chem. Soc. 134(10).4731-4742 (2012)) who showed this structure sequesters the protein Muscleblind Like Splicing Regulator 1 (MBNL1), an RNA splicing protein that in humans is encoded by the MBNL1 gene. MBNL1 has a well characterized role in myotonic dystrophy where impaired RNA splicing disrupts muscle development and function.

### Measurement of Direct RNA Repeat Binding

The method used for the MST fluorescence assay was as follows. A serial dilution of the ligand (Tideglusib) was prepared in a way to match the final buffer conditions in the reaction mix (assay buffer 20 mM HEPES pH 7.4, 0.05% Tween-20). The highest concentration of ligand was 500 μM and the lowest 15.3 nM. 5 μl of each dilution step were mixed with 5 μl of the fluorescent molecule. The final reaction mixture, which was filled in capillaries, contained a respective amount of ligand (max. conc. 250 μM, min. conc. 7.63 nM) and constant 50 nM fluorescent molecule. The samples were analyzed on a Monolith NT. 115 at 25° C., with 40% LED power and 40% Laser power. The fluorescence labeled RNA construct (RNA hairpin 2; produced by Metabion) had the following sequence:

RNA hairpin 1:
(SEQ ID NO: 1)
Cy5-ggg aga ggg uuu aau cug cug cug cug cug cug
uac gaa agu acu gcu gcu gcu gcu gcu gau ugg auc
cgc aag g-3

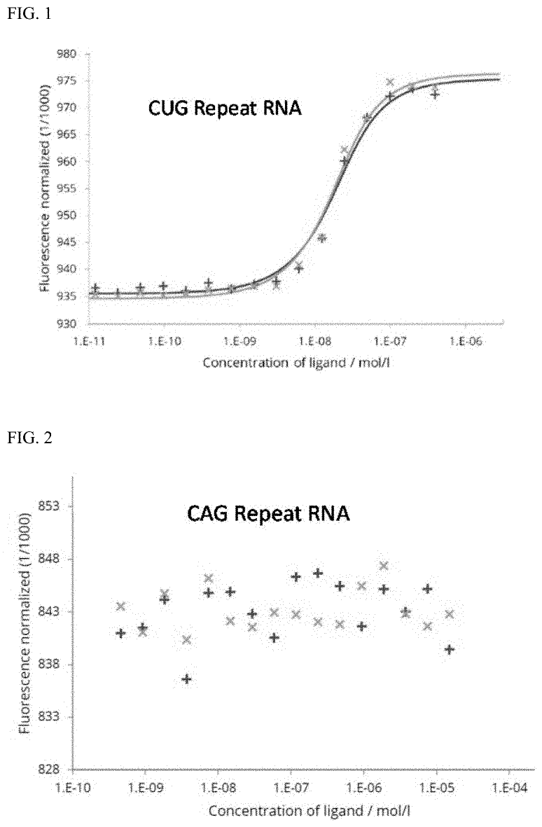

The data obtained showed a dose dependent direct binding of Tideglusib to this construct (FIG. 1) with a K_{D} of 19.7 nM.

Binding to other RNA trinucleotide repeats was investigated by repeating the experimental protocol above with an RNA repeat construct containing CAG repeats (RNA hairpin 2).

RNA hairpin 2:
(SEQ ID NO: 2)
Cy5-ggg aga ggg uuu aau cag cag cag cag cag cag
uac gaa agu aca gca gca gca gca gca gau ugg auc
cgc aag g-3

The data obtained showed no reliable binding of Tideglusib to this construct (FIG. 2).

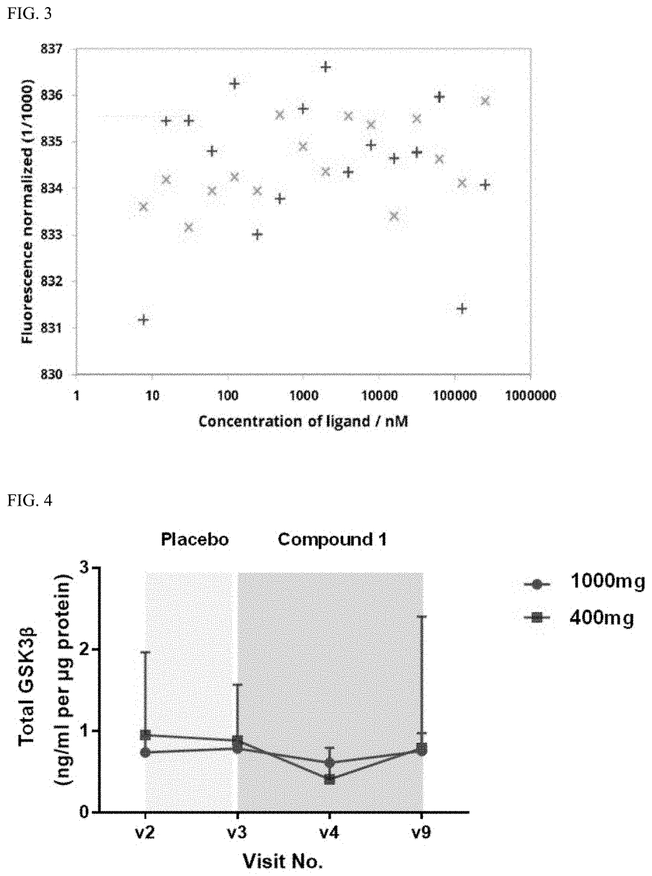

Other substituted thiadiazolidines tested also showed direct binding to repeat RNAs. 2-Benzyl-4-ethyl-[1,2,4]thiadiazolidine-3,5-dione (Compound 2) bound to RNA hairpin 1 with a K_{D} of 31.4 μM (data not shown). However, a urea derivative of Tideglusib (1-benzyl-3-naphthalen-1-yl-urea) had no binding activity (FIG. 3).

Inhibition of GSK3β in Tissues from Patients Treated with Tideglusib

In order to determine whether the clinical benefit produced by administration of Tideglusib could be related to inhibition of GSK3β, the activity of this kinase in circulating tissues in treated patients was determined. Lymphocyte cell pellets were lysed in 150 μl of cold NP40 Cell Lysis Buffer with protease inhibitors. Total protein of each extract was measured with DC Protein assay Kit according to the manufacturer specifications. For the analysis of GSK3β and related kinases, the commercially available kits AKT Pathway Total Multispecies 7-Plex Panel and the equivalent for the phosphorylated forms for Luminex® Platform from Thermo Fisher Scientific were used. These were the AKT Pathway (Total) 7-Plex Multispecies Panel (#LHO0002M ThermoFisher Scientific) that assays the following signaling proteins: GSK3β, Total IR, IGF-1R, IRS-1, Akt, PRAS40 and p70s6K. The AKT Pathway (Phospho) 7-Plex Multispecies Panel (#LHO0001M ThermoFisher Scientific) assays the following signaling proteins: GSK3β[pS9], IR[pYpY1162/1163], IGF-1R[pYpY1135/1136], IRS-1[pS312], Akt[pS473], PRAS40[pT246] and p70s6K[pTpS421/424].

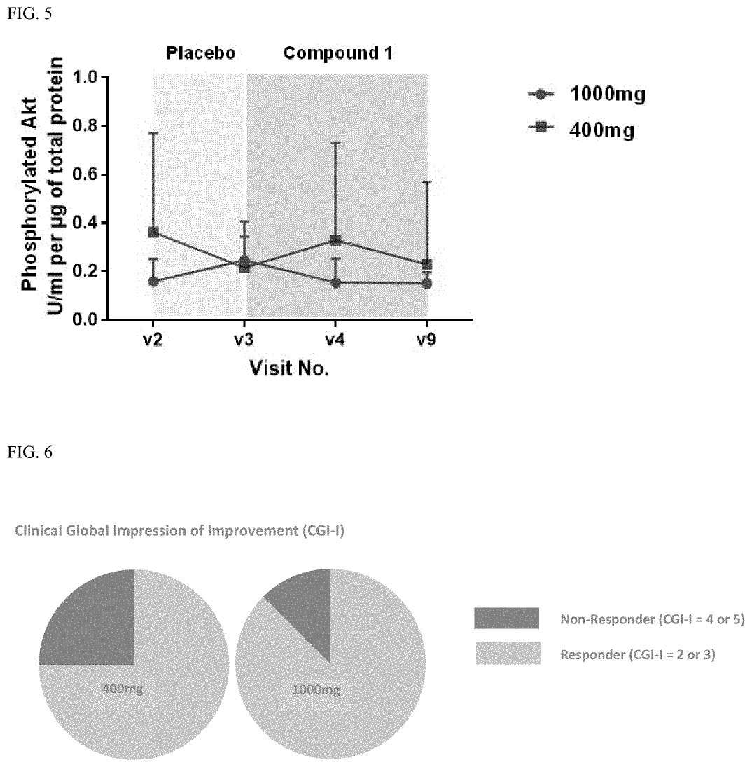

FIG. 4 shows the levels of inhibition of GSK3β in lymphocytes from patients during treatment with Tideglusib, with doses of 400 mg po per day and 1000 mg po per day. Neither dose shows any effect of Tideglusib on GSK3β levels. GSK3β is a kinase responsible for phosphorylation of the downstream kinase Akt, therefore successful in vivo inhibition of GSK3β should reduce levels of phosphorylation of Akt. FIG. 5 shows this is not achieved in patients with DM1 myotonic dystrophy treated with Tideglusib in the dose range up to 1000 mg per day. However, as shown in FIG. 6, the majority of patients having DM1 treated with Tideglusib showed clinical benefit as assessed using the Clinical Global Impression of Improvement measurement scale.

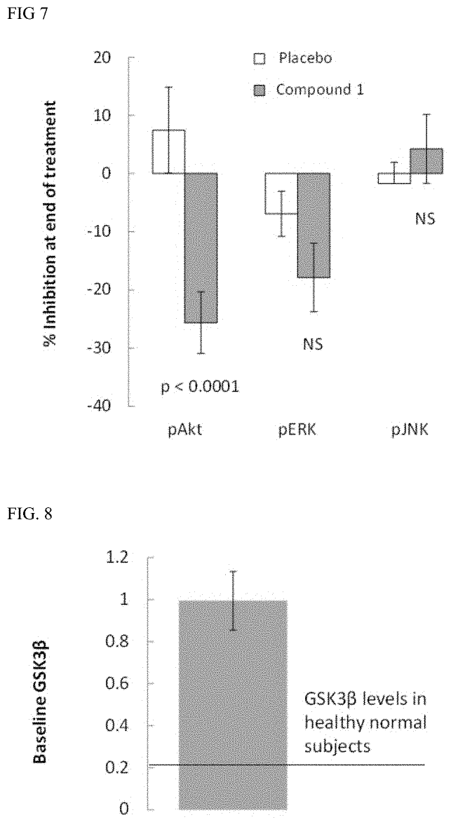

These data differ from those obtained upon administration of Tideglusib to other patient groups. FIG. 7 shows the effect of administration of Tideglusib to patients with Autism Spectrum Disorder. In these patients, administration of Tideglusib reduces levels of phosphorylation of Akt, as would be predicted if in vivo inhibition of the activity of GSK3β was occurring. The difference in response in relation to the ability of Tideglusib to inhibit GSK3β in vivo in patients may be accounted for by differing baseline levels of GSK3β activity. In patients with DM1 myotonic dystrophy, baseline levels of GSK3β kinase are known to be elevated above normal (FIG. 8; Jones et al., J. Clin. Invest. 122(12):4461-4472 (2012)). These data show that excess amounts of GSK3β kinase protein are present in DM1 patient tissues. This finding is not shown in patients with Autism Spectrum Disorder. The data therefore show that Tideglusib administered in the dose range 400 mg to 1000 mg is unable overcome the phosphorylation of Akt seen in the presence of elevated levels of GSK3β, in contrast to the effect of Tideglusib seen in the presence of normal levels of GSK3β in Autism Spectrum Disorder patients.

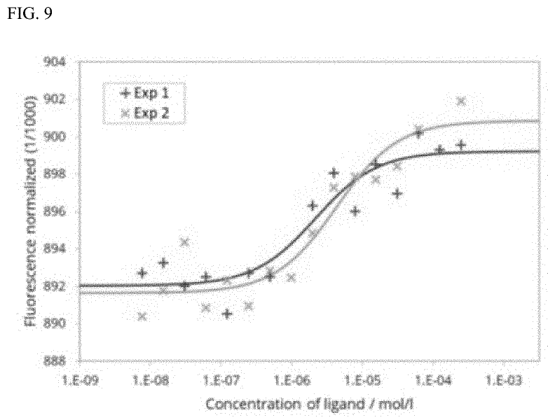

Importantly, the binding of Tideglusib to a CUG repeat RNA construct is modulated by the presence of non-CUG repeat interruptions in the sequence of the construct used in testing (RNA hairpin 3; SEQ ID NO:3). Interspersed non CUG repeats (CCG and CUC) reduced the potency of binding of Tideglusib to 3.2 μM (FIG. 9). That is binding was 162 fold less potent versus biding seen when these additional repeats were not added as shown above with RNA hairpin 1.

RNA hairpin 3:
(SEQ ID NO: 3)
Cy5-ggg aga ggg uuu aau cug cug cug cug cug ccg
cug cug cug cug cug cuc cug cug cug cug cug ccg
cug cug cug cug cug cuc uac gaa agu ag cug cug
cug cug cug gcg cug cug cug cug cug cgg cug cug
cug cug cug gcg cug cug cug cug cug cga uug gau
ccg caa gg-3

Tideglusib appears therefore to bind most potently in the absence of interrupting non CUG repeats that are known to reduce the pathogenic consequences of CUG repeat RNAs and produce less severe clinical symptoms. Tideglusib may prove to reduce symptoms of DM1 most effectively in patients who do not have non CUG repeat interruptions in their CUG repeat RNA and have most severe symptoms. However, Tideglusib is also expected to be effective in the treatment of the Adult Onset DM1 patients with no or few non CUG RNA repeat interruptions and worse symptoms.

What is claimed is:

1. A method of treating a disease associated with a RNA molecule having an abnormal repeat sequence in a subject, comprising administering to a subject in need thereof a therapeutically-effective amount of a compound of Formula I or a pharmaceutically acceptable salt, prodrug or solvate thereof.

2. A method of preventing a disease associated with a RNA molecule having an abnormal repeat sequence in a subject, comprising administering to a subject in need thereof a therapeutically-effective amount of a compound of Formula I or a pharmaceutically acceptable salt, prodrug or solvate thereof.

3. A method of inhibiting a RNA molecule having an abnormal repeat sequence with a compound of Formula I or a pharmaceutically acceptable salt, prodrug or solvate thereof, comprising contacting a RNA molecule having an abnormal repeat sequence with a compound of Formula I or a pharmaceutically acceptable salt, prodrug or solvate thereof.

4. The method of claim 1, wherein the compound of Formula I is a compound of: 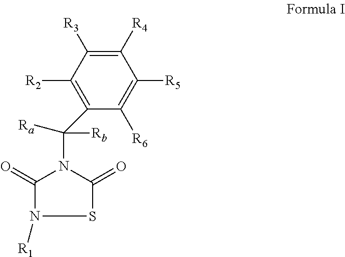
wherein:
R₁ is an organic group having at least 8 atoms selected from C or O, which is not linked directly to the N through a —C(O)— and comprising at least an aromatic ring;
Rₐ, R_{b}, R₂, R₃, R₄, R₅, R₆ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₇, —C(O)OR₇, —C(O)NR₇R₈ —C═NR₇, —CN, —OR₇, —OC(O)R₇, —S(O)ₜ—R₇, —NR₇R₈, —NR₇C(O)R₈, —NO₂, —N═CR₇R₈ or halogen;
t is 0, 1, 2 or 3;
R₇ and R₈ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;
wherein Rₐ and R_{b} together can form a group ═O, and wherein any pair Rₐ R₂, R₂ R₃, R₃ R₄, R₄ R₅, R₅ R₆, R₆ R_{b}, or R₇R₈ can form together a cyclic substituent;
or a pharmaceutically acceptable salt, prodrug or solvate thereof.

5. The method of claim 1, wherein the compound of Formula I is selected from the group consisting of: 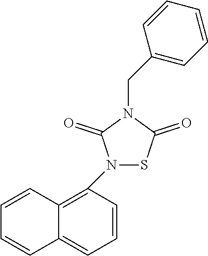 [SMILES: O=c1sn(-c2cccc3ccccc23)c(=O)n1Cc1ccccc1]
4-Benzyl-2-naphthalen-1-yl-[1,2,4]thiadiazolidine-3,5-dione (Tideglusib) 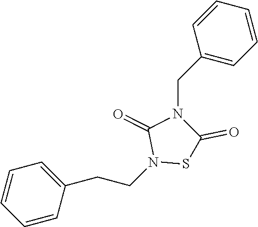 [SMILES: O=c1sn(CCc2ccccc2)c(=O)n1Cc1ccccc1]
4-Benzyl-2-phenethyl-[1,2,4]thiadiazolidine-3,5-dione 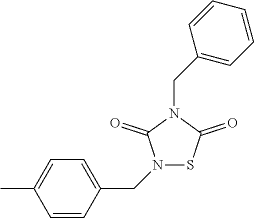 [SMILES: Cc1ccc(Cn2sc(=O)n(Cc3ccccc3)c2=O)cc1]
4-Benzyl-2-(4-methyl-benzyl)-[1,2,4]thiadiazolidine-3,5-dione 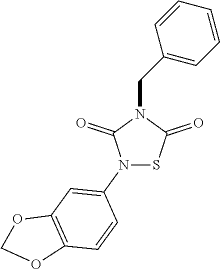
2-Benzo[1,3]dioxol-5-yl-4-benzyl-[1,2,4]thiadiazolidine-3,5-dione 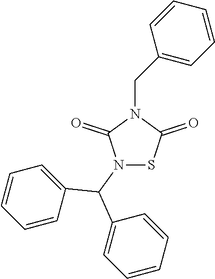 [SMILES: O=c1sn(C(c2ccccc2)c2ccccc2)c(=O)n1Cc1ccccc1]
4-Benzyl-2-diphenylmethyl-[1,2,4]-thiadiazolidine-3,5-dione 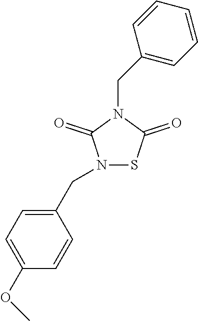 [SMILES: COc1ccc(Cn2sc(=O)n(Cc3ccccc3)c2=O)cc1]
4-Benzyl-2-(4-methoxy-benzyl)-[1,2,4]thiadiazolidine-3,5-dione 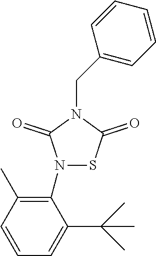 [SMILES: Cc1cccc(C(C)(C)C)c1-n1sc(=O)n(Cc2ccccc2)c1=O]
4-Benzyl-2-(2-tert-butyl-6-methyl-phenyl)-[1,2,4]thiadiazolidine-3,5-dione 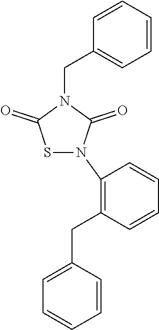 [SMILES: O=c1sn(-c2ccccc2Cc2ccccc2)c(=O)n1Cc1ccccc1]
4-Benzyl-2-(2-benzyl-phenyl)-[1,2,4]thiadiazolidine-3,5-dione, and 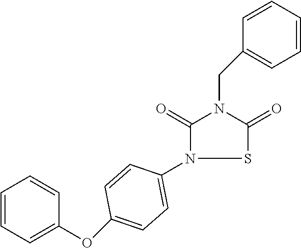 [SMILES: O=c1sn(-c2ccc(Oc3ccccc3)cc2)c(=O)n1Cc1ccccc1]
4-Benzyl-2-(4-phenoxy-phenyl)-[1,2,4]thiadiazolidine-3,5-dione,
or a pharmaceutically acceptable salt, prodrug or solvate thereof.

6. The method of claim 1, wherein the compound of Formula I is Tideglusib (4-Benzyl-2-naphthalen-1-yl-[1,2,4]thiadiazolidine-3,5-dione) or a pharmaceutically acceptable salt, prodrug or solvate thereof.

7. The method of claim 2, wherein the compound of Formula I is a compound of: 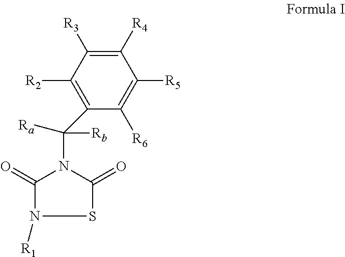
wherein:
R₁ is an organic group having at least 8 atoms selected from C or O, which is not linked directly to the N through a —C(O)— and comprising at least an aromatic ring;
Rₐ, R_{b}, R₂, R₃, R₄, R₅, R₆ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₇, —C(O)OR₇, —C(O)NR₇R₈ —C═NR₇, —CN, —OR₇, —OC(O)R₇, —S(O)ₜ—R₇, —NR₇R₈, —NR₇C(O)R₈, —NO₂, —N═CR₇R₈ or halogen;
t is 0, 1, 2 or 3;
R₇ and R₈ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;
wherein Rₐ and R_{b} together can form a group ═O, and wherein any pair Rₐ R₂, R₂ R₃, R₃ R₄, R₄ R₅, R₅ R₆, R₆ R_{b}, or R₇R₈ can form together a cyclic substituent;
or a pharmaceutically acceptable salt, prodrug or solvate thereof.

8. The method of claim 2, wherein the compound of Formula I is selected from the group consisting of: 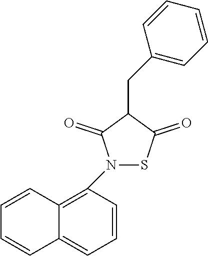 [SMILES: O=C1SN(c2cccc3ccccc23)C(=O)C1Cc1ccccc1]
4-Benzyl-2-naphthalen-1-yl-[1,2,4]thiadiazolidine-3,5-dione (Tideglusib) 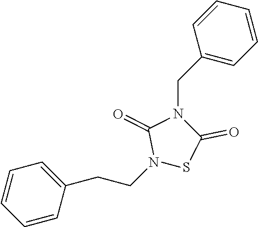 [SMILES: O=c1sn(CCc2ccccc2)c(=O)n1Cc1ccccc1]
4-Benzyl-2-phenethyl-[1,2,4]thiadiazolidine-3,5-dione 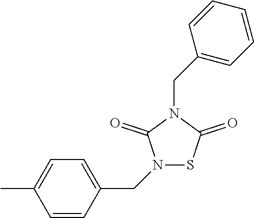 [SMILES: Cc1ccc(Cn2sc(=O)n(Cc3ccccc3)c2=O)cc1]
4-Benzyl-2-(4-methyl-benzyl)-[1,2,4]thiadiazolidine-3,5-dione 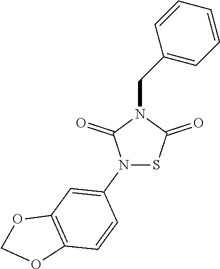
2-Benzo[1,3]dioxol-5-yl-4-benzyl-[1,2,4]thiadiazolidine-3,5-dione 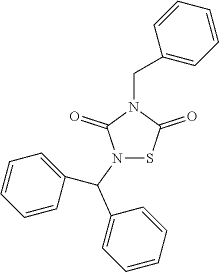 [SMILES: O=c1sn(C(c2ccccc2)c2ccccc2)c(=O)n1Cc1ccccc1]
4-Benzyl-2-diphenylmethyl-[1,2,4]-thiadiazolidine-3,5-dione 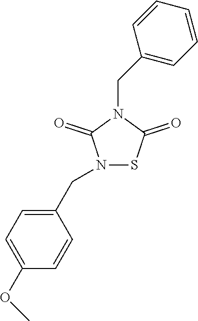 [SMILES: COc1ccc(Cn2sc(=O)n(Cc3ccccc3)c2=O)cc1]
4-Benzyl-2-(4-methoxy-benzyl)-[1,2,4]thiadiazolidine-3,5-dione 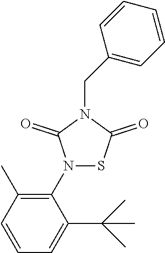 [SMILES: Cc1cccc(C(C)(C)C)c1-n1sc(=O)n(Cc2ccccc2)c1=O]
4-Benzyl-2-(2-tert-butyl-6-methyl-phenyl)-[1,2,4]thiadiazolidine-3,5-dione 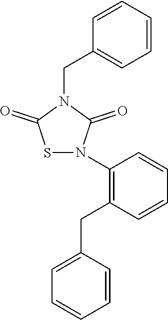 [SMILES: O=c1sn(-c2ccccc2Cc2ccccc2)c(=O)n1Cc1ccccc1]
4-Benzyl-2-(2-benzyl-phenyl)-[1,2,4]thiadiazolidine-3,5-dione, and 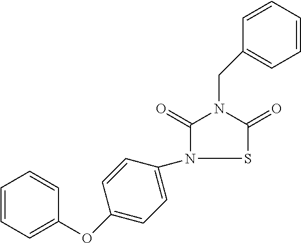 [SMILES: O=c1sn(-c2ccc(Oc3ccccc3)cc2)c(=O)n1Cc1ccccc1]
4-Benzyl-2-(4-phenoxy-phenyl)-[1,2,4]thiadiazolidine-3,5-dione,
or a pharmaceutically acceptable salt, prodrug or solvate thereof.

9. The method of claim 2, wherein the compound of Formula I is Tideglusib (4-Benzyl-2-naphthalen-1-yl-[1,2,4]thiadiazolidine-3,5-dione) or a pharmaceutically acceptable salt, prodrug or solvate thereof.

10. The method of claim 3, wherein the compound of Formula I is a compound of: 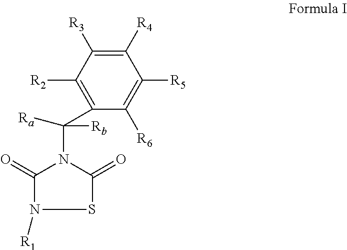
wherein:
R₁ is an organic group having at least 8 atoms selected from C or O, which is not linked directly to the N through a —C(O)— and comprising at least an aromatic ring;
Rₐ, R_{b}, R₂, R₃, R₄, R₅, R₆ are independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, —COR₇, —C(O)OR₇, —C(O)NR₇R₈ —C═NR₇, —CN, —OR₇, —OC(O)R₇, —S(O)ₜ—R₇, —NR₇R₈, —NR₇C(O)R₈, —NO₂, —N═CR₇R₈ or halogen;
t is 0, 1, 2 or 3;
R₇ and R₈ are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, halogen;
wherein Rₐ and R_{b} together can form a group ═O, and wherein any pair Rₐ R₂, R₂ R₃, R₃ R₄, R₄ R₅, R₅ R₆, R₆ R_{b}, or R₇R₈ can form together a cyclic substituent;
or a pharmaceutically acceptable salt, prodrug or solvate thereof.

11. The method of claim 3, wherein the compound of Formula I is selected from the group consisting of: 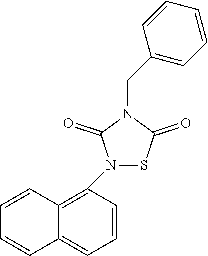 [SMILES: O=c1sn(-c2cccc3ccccc23)c(=O)n1Cc1ccccc1]
4-Benzyl-2-naphthalen-1-yl-[1,2,4]thiadiazolidine-3,5-dione (Tideglusib) 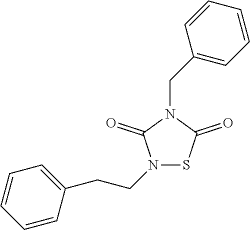 [SMILES: O=c1sn(CCc2ccccc2)c(=O)n1Cc1ccccc1]
4-Benzyl-2-phenethyl-[1,2,4]thiadiazolidine-3,5-dione 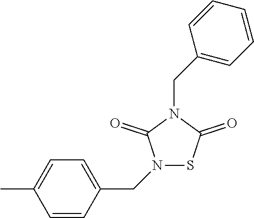 [SMILES: Cc1ccc(Cn2sc(=O)n(Cc3ccccc3)c2=O)cc1]
4-Benzyl-2-(4-methyl-benzyl)-[1,2,4]thiadiazolidine-3,5-dione 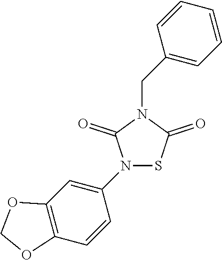 [SMILES: O=c1sn(-c2ccc3c(c2)OCO3)c(=O)n1Cc1ccccc1]
2-Benzo[1,3]dioxol-5-yl-4-benzyl-[1,2,4]thiadiazolidine-3,5-dione 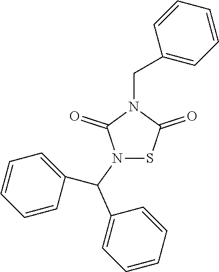 [SMILES: O=c1sn(C(c2ccccc2)c2ccccc2)c(=O)n1Cc1ccccc1]
4-Benzyl-2-diphenylmethyl-[1,2,4]-thiadiazolidine-3,5-dione 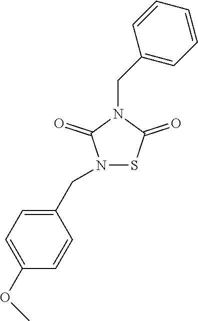 [SMILES: COc1ccc(Cn2sc(=O)n(Cc3ccccc3)c2=O)cc1]
4-Benzyl-2-(4-methoxy-benzyl)-[1,2,4]thiadiazolidine-3,5-dione 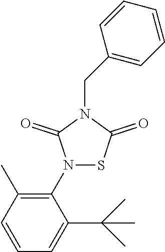 [SMILES: Cc1cccc(C(C)(C)C)c1-n1sc(=O)n(Cc2ccccc2)c1=O]
4-Benzyl-2-(2-tert-butyl-6-methyl-phenyl)-[1,2,4]thiadiazolidine-3,5-dione 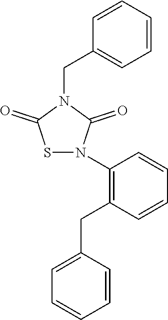 [SMILES: O=c1sn(-c2ccccc2Cc2ccccc2)c(=O)n1Cc1ccccc1]
4-Benzyl-2-(2-benzyl-phenyl)-[1,2,4]thiadiazolidine-3,5-dione, and 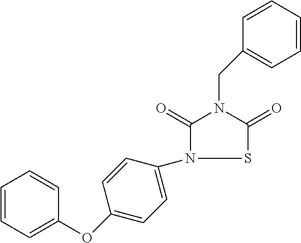 [SMILES: O=c1sn(-c2ccc(Oc3ccccc3)cc2)c(=O)n1Cc1ccccc1]
4-Benzyl-2-(4-phenoxy-phenyl)-[1,2,4]thiadiazolidine-3,5-dione,
or a pharmaceutically acceptable salt, prodrug or solvate thereof.

12. The method of claim 3, wherein the compound of Formula I is Tideglusib (4-Benzyl-2-naphthalen-1-yl-[1,2,4]thiadiazolidine-3,5-dione) or a pharmaceutically acceptable salt, prodrug or solvate thereof.

13. The method of claim 1, wherein the disease associated with a RNA molecule having an abnormal repeat sequence is selected from the group consisting of Congenital myotonic dystrophy type 1 (DM1), Childhood DM1, Adult Onset DM1, Fuchs endothelial corneal dystrophy and spinocerebellar ataxia type 8.

14. The method of claim 2, wherein the disease associated with a RNA molecule having an abnormal repeat sequence is selected from the group consisting of Congenital myotonic dystrophy type 1 (DM1), Childhood DM1, Adult Onset DM1, Fuchs endothelial corneal dystrophy and spinocerebellar ataxia type 8.

15. The method of claim 1, wherein the RNA molecule having an abnormal repeat sequence is a RNA molecule comprising one or more consecutive and uninterrupted CUG nucleotide repeat sequences of at least 50 CUG nucleotide repeats.

16. The method of claim 2, wherein the RNA molecule having an abnormal repeat sequence is a RNA molecule comprising one or more consecutive and uninterrupted CUG nucleotide repeat sequences of at least 50 CUG nucleotide repeats.

17. The method of claim 3, wherein the RNA molecule having an abnormal repeat sequence is a RNA molecule comprising one or more consecutive and uninterrupted CUG nucleotide repeat sequences of at least 50 CUG nucleotide repeats.

18. The method of claim 1, wherein the compound of Formula I is formulated in a pharmaceutical composition comprising a compound of Formula I and one or more pharmaceutically acceptable excipient, carrier, adjuvant and/or vehicle.

19. The method of claim 2, wherein the compound of Formula I is formulated in a pharmaceutical composition comprising a compound of Formula I and one or more pharmaceutically acceptable excipient, carrier, adjuvant and/or vehicle.

20. The method of claim 3, wherein the compound of Formula I is formulated in a pharmaceutical composition comprising a compound of Formula I and one or more pharmaceutically acceptable excipient, carrier, adjuvant and/or vehicle.

21. The method of claim 18, wherein the pharmaceutical composition is formulated for oral delivery.

22. The method of claim 19, wherein the pharmaceutical composition is formulated for oral delivery.

23. The method of claim 20, wherein the pharmaceutical composition is formulated for oral delivery.

24. A method of treating myotonic dystrophy type 1 (DM1) in a subject, comprising administering to a subject in need thereof a therapeutically-effective amount of Tideglusib (4-Benzyl-2-naphthalen-1-yl-[1,2,4]thiadiazolidine-3,5-dione) or a pharmaceutically acceptable salt, prodrug or solvate thereof.

25. The method of claim 24, wherein DM1 is Adult Onset DM1 or Congenital DM1.

26. The method of claim 1, wherein the therapeutically-effective amount of a compound of Formula I or a pharmaceutically acceptable salt, prodrug or solvate thereof ranges from 300 to 1000 mg, administered once per day or once every two days.

27. The method of claim 2, wherein the therapeutically-effective amount of a compound of Formula I or a pharmaceutically acceptable salt, prodrug or solvate thereof ranges from 300 to 1000 mg, administered once per day or once every two days.

29. The method of claim 3, wherein the therapeutically-effective amount of a compound of Formula I or a pharmaceutically acceptable salt, prodrug or solvate thereof ranges from 300 to 1000 mg, administered once per day or once every two days.

29. The method of claim 24, wherein the therapeutically-effective amount of a compound of Formula I or a pharmaceutically acceptable salt, prodrug or solvate thereof ranges from 300 to 1000 mg, administered once per day or once every two days.


SEQUENCE LISTING (3 sequences)

SEQ ID NO: 1 - RNA, 76 nt, Artificial Sequence
  chemically synthesized RNA hairpin 1
gggagaggguuuaaucugcugcugcugcugcuguacgaaaguacugcugcugcugcugcugauuggauccgcaagg

SEQ ID NO: 2 - RNA, 76 nt, Artificial Sequence
  chemically synthesized RNA hairpin 2
gggagaggguuuaaucagcagcagcagcagcaguacgaaaguacagcagcagcagcagcagauuggauccgcaagg

SEQ ID NO: 3 - RNA, 184 nt, Artificial Sequence
  chemically synthesized RNA hairpin 3
gggagaggguuuaaucugcugcugcugcugccgcugcugcugcugcugcuccugcugcugcugcugccgcugcugcugcugcugcucuacgaaaguagcugcugcugcugcuggcgcugcugcugcugcugcggcugcugcugcugcuggcgcugcugcugcugcugcgauuggauccgcaagg